(19) United States
(12) Patent Application Publication
(10) Pub. No.: US 2014/0328895 A1
(43) Pub. Date: Nov. 6, 2014

(54) FILM-FORMING COMPOSITION FOR A PH-DEPENDANT SUSTAINED RELEASE OF THE ACTIVE AGENT

(71) Applicants: Michael Friedman, Jerusalem (IL); Doron Steinberg, Jerusalem (IL); Eran Lavy, Mobile Post Shikmim (IL)

(72) Inventors: Michael Friedman, Jerusalem (IL); Doron Steinberg, Jerusalem (IL); Eran Lavy, Mobile Post Shikmim (IL)

(73) Assignee: Yissum Research Development Company of the Hebrew University of Jerusalem, Ltd., Jerusalem (IL)

(21) Appl. No.: 14/007,347

(22) PCT Filed: Mar. 25, 2012
(86) PCT No.: PCT/IL12/00129

§ 371 Date: Jul. 25, 2014

Related U.S. Application Data
(60) Provisional application No. 61/468,709, filed on Mar. 29, 2011.
(60) Provisional application No. 61/476,928, filed on Apr. 19, 2011.

(51) Int. Cl. A61L 29/08; A61L 31/16; A61K 31/4425; A61K 47/32; A61K 31/09; A61L 29/16; A61K 47/38; A61K 47/10; A61K 31/155; A61K 31/4174

(52) U.S. Cl. CPC A61L 29/085; A61K 31/155; A61L 31/16; A61K 31/4425; A61K 31/4174; A61K 31/09; A61L 29/16; A61K 47/38; A61K 47/10; A61K 47/32; A61L 2420/06; A61L 2420/02
USPC 424/430; 514/635; 424/422; 514/358; 514/396; 514/721; 427/2.3

(57) ABSTRACT

The present invention discloses a liquid precursor composition adapted for application on a on a desired surface, this composition comprising: a. at least one therapeutic agent suitable for the treatment or prevention of a disorder or pathological condition, wherein said disorder or pathological condition excludes oral disorders, b. at least one acidic-pH sensitive polymer, c. at least one hydrophobic polymer, and d. a pharmaceutically acceptable volatile solvent, wherein a weight ratio between the at least one hydrophobic polymer and the at least one acidic-pH sensitive polymer is larger than 1.

Sustained release delivery (SRD) systems are pharmaceutical applications in which the active agent is released from the vehicle at a controlled rate.

Several pharmacological advantages stem from the use of SRD: controlled duration and concentrations of the drug in the target site; reduced amount of applied drug and minimal side effects (such as bitter taste, tooth staining, the development of resistant bacterial strains, and the recurrence of oral infections). These advantages in turn result in better clinical improvement and better patient compliance.

Sustained release delivery systems have indeed been reported to be useful in some cases for the local treatment of periodontal disease and in the treatment of plaque prevention in patients wearing orthodontic appliances (see for example, Friedman, M., et al., J. Dent. Res. 64:1319-1321, 1985). In this system, the active ingredient was embedded in an ethyl cellulose polymer to form a film. U.S. Pat. No. 5,330,746 by the present inventors discloses dental liquid precursor compositions for plaque prevention or for treating and/or preventing tooth hypersensitivity, whereas the antibacterial agent or the hypersensitivity agent were embedded in a sustained release carrier, such as a hydrophilic polymer, an acrylic polymer, or a combination of both.

U.S. Pat. No. 5,160,737 by some of the present inventors shows that acrylic polymers can be used as a matrix for sustained release of agents such as cetylpyridium-chloride (CPC).

In addition, biofilm formation, which is associated with change of pH, is also associated with other oral disorders such as hypersensitive teeth and tooth staining and oral ulceration.

In addition, most of the inflammation process are subject to pH change pending on the type of microbes and surrounding environment.

In addition, the release rate can be monitored externally by the control of the pH of the environment which the SRV is replaced in.

The use of medical devices inserted into a patient's body is now routine in healthcare management within hospitals and nursing homes. Although there are substantial benefits associated with the use of inserted medical devices, such as, for example, catheters and stents, there are very worryingly a number of potentially dangerous complications that may lead to an increase in the time patients remain in hospital and more importantly in an increase in the number of patient deaths associated with the use of these devices. These complications arise principally because of the way in which a patient's body reacts to insertion of a medical device and what it perceives to be a foreign object. Consequently patients are often plagued by infection associated with the insertion of a medical device and this is seen to be one of the most critical disadvantages of an otherwise highly effective and beneficial medical treatment. There is an urgent need to improve what is often referred to as device-related infection.

Typically device-related infection begins with bacterial/fungi/virus adherence, developing with the formation of biofilm. Bacteria and other pathogens which typically colonize catheters produce urease which degrades urea in urine to form carbon dioxide and ammonia. At increased pH associated with such degradation, minerals in urine precipitate leading to encrustation

WO 2010/0264333 discloses a device (such as a stent) comprising a body structure, having one or more surfaces which are composed of a pH sensitive layer, that has a changing water solubility at a pH trigger. This device was used to prevent infection when the physiological pH around the device changed, for example due to bacterial infection.

### SUMMARY OF THE INVENTION

The inventors have now successfully developed a sustained release formulation in which the release rate is controlled by change of pH thereby releasing larger amounts of the therapeutic agent when needed—i.e when there is a disorder or pathological condition associated with a change in pH as decrease in pH, or when the condition deteriorates (evidenced by decreased pH); while at times of remission, where the pH returns to normal, the release rate will return to the basal release rate.

The proposed system is unique in that it has a “built-in” pH sensor that controls the release rate due to pH changes. E.g.; increases the release rate at acidic pH.

In particular, the present invention describes a liquid precursor composition adapted for application on a desired surface, comprising:
a) at least one therapeutic agent suitable for the treatment or prevention of a disorder or pathological condition; such that this disorder or pathological condition excludes oral disorders,
b) at least one acidic-pH sensitive polymer
c) at least one hydrophobic polymer; and
d) a pharmaceutically acceptable volatile solvent,
wherein a weight ratio between the at least one hydrophobic polymer and the at least one acidic-pH sensitive polymer is larger than 1.

By one aspect of the invention the “desired” surface is the surface of a medical device that is to be inserted in or placed on a body of a subject (a human or an animal).

The medical device can be used to deliver also drugs systemically by adsorption via the skin or mucosa thereby passing the GI track using molecules with first path degradation effect when absorbed from GI track.

Examples of such medical devices are: catheters (including urinary catheters), stents (including urinary stents), defibrillators, pacemakers, pumps, electrodes, artificial joints, air-tubes, CVS, implants, heart valves, intrauterine devices, artificial joints, implants, feeding tubes, ventilation tubes, IV's and discharge tubes.

Typically the composition of the invention is applied on the device in order to prevent infection from forming, or treat an established infection. The infection may be bacterial, viral, fungal or protozoa and may be due also to biofilm formation of bacteria or fungi. Consequently typically the active agent is an antibiotic, an antifungal, an anti-viral, an antiprotozoa, an antiseptic, anti inflammatory, anti irritation, anti pain, local analgestic, anti histamines, hormones, enzymes, anti cancer, anastetic, peptides antibiofilm, anti quorum sensing, genes or plasmids agent.

By another aspect the surface is an external surface of body tissue. Examples of such body tissues include, but are not limited to, soft tissues, skin, mucosa (excluding oral mucosa), nails, hoofs and udder (animals and or human).

Typically the composition of the invention is applied to the external surface in order to prevent infection from forming, or treat an established infection. The infection may be bacterial, viral, fungal or protozoa and may be due also to biofilm formation of bacteria or fungi. Consequently typically the active agent is antibiotic, antifungal, anti-viral, antiprotozoa, antiseptic or anti-inflammatory agent, anti irritation, anti pain, local analgestic, anti histamines, hormones, enzymes, anti cancer, peptidesor, antibiofilm, anti quorum sensing, genes or plasmids agents.

The varnish is applied on the desired surface, such as any tissues (human and/or animal) internal or external such as skin, soft tissues, hard tissues mucosa (excluding oral mucosa) directly by means as brushing, coating or spraying.

By a specific example the composition is applied to treat the following conditions: on mucosa to treat any infections and disorders also internal mucosa as in the case of cystitis, on skin to treat infection by microorganism and disorders resulting from these infections. On udder (to treat disorders as mastitis), on nails (to treat disorders as fungi infections) and on hoofs.

The term “liquid” refers to a composition which is fluid at room temperature when present in the vessel.

The term “liquid precursor” means that while the composition of the invention is initially liquid, upon application to a surface it solidifies (mainly due to evaporation of the pharmaceutically acceptable volatile carrier or solvent).

The solvent (at times referred to as “carrier”) is usually a biocompatible and volatile (at body temperature) solvent.

The solvent suitable to be used as part of the liquid precursor composition of the present invention, should be capable of evaporating under physiological conditions (after the device is inserted or placed in the body of the subject or after the composition is administered to an external surface of the subject.

Preferably, the solvent is an alcohol or a combination of alcohol and water (a hydro-alcoholic or alcoholic solvent).

More preferably, the solvent is selected from the group consisting of ethyl alcohol or a combination of ethyl alcohol and water or any other solvent that is biocompatible and not toxic.

The term “adapted for application to a surface” refers to the fact that the liquid precursor composition is to be applied (by brushing, dipping, spraying etc on the surface as described above—which may be the surface of a medical device or an external surface of a body.

The term “acidic-pH sensitive polymer” refers to a biocompatible polymer that increases its solubility at acidic pH. The term “acidic pH” as used herein refers to the pH decreasing below the decreases from the normal pH of 7.2-6.8. More preferably, the acidic-pH sensitive polymer would have an enhanced solubility at about or below pH 6.0.

An example of an acidic pH sensitive polymer is dimethylaminoethyl methacrylate copolymer (Eudragit E). Other acrylate polymers of the Eudragit family and or other polymers containing primary, secondary or tertiary amine groups may be used for this purpose.

Thus, according to one preferred embodiment of the invention, the acidic-pH sensitive polymer is selected from Eudragit E, acrylic compounds or any compounds containing primary, secondary or tertiary amine groups.

According to a preferred embodiment of the invention, the acidic-pH sensitive polymer forms between 10% by weight to 40% by weight of the total weight of the matrix.

The term “hydrophobic polymer” refers to a biocompatible polymer having hydrophobic properties, which is further non-soluble under physiological conditions.

Non-limiting examples of hydrophobic polymers include, but are not limited to the following polymers, as well as their cross-linked versions e.g. aldehydes or polar compounds) and chemical derivatives: copolymer hydrogels of hydroxymethyl methacrylate (HEMA) and methylmethacrylate (MMA), Ethyl cellulose (EC), Silicone rubber, polyethylene, poly(ethylene oxide), poly(acrylic acid), polylactic acid, polymethylmethacrylate, poly(methyl vinyl ether co-maleic anhydride), poly(hydroxyethylmethacrylate), polyvinyl chloride, polyurethane, polyvinyl acetate, cellulose nitrate, karya gum, ethylvinyl acetate, polystyrene, polyamide and proteins.

Preferably, the hydrophobic polymer is selected from copolymer hydrogels of hydroxymethyl methacrylate (HEMA) and methylmethacrylate (MMA), Ethyl cellulose (EC), poly(acrylic acid), poly(methyl vinyl ether co-maleic anhydride), poly(ethylene oxide), karya gum, poly(hydroxyethylmethacrylate), Silicone rubber, polyethylene, polylactic acid, polymethylmethacrylate, polyvinyl chloride, polyvinyl acetate, and polyurethane.

More preferably, the at least one hydrophobic polymer is selected from: cross linked polymers and derivatives of polymers such as Ethyl cellulose, Silicone rubber, polyethylene, polylactic acid, polymethylmethacrylate, polyvinyl chloride, polyurethane.

In a preferred embodiment of the present invention, the composition comprises Ethyl Cellulose as the hydrophobic polymer and Eudragit E as the acidic pH sensitive polymer.

Generally, the range of the hydrophobic polymer would be from about 30% to about 80%, the pH sensitive polymer ranging from about 10% to about 30%, and the active agent ranging from about 5% to about 40%, all of these in the dry film.

However, in order to achieve the beneficial properties of the present composition, it is important that the ratio between the hydrophobic polymer and the acidic-pH sensitive polymer is kept higher than 1. This will ensure that upon solidification of the liquid precursor composition, the hydrophobic polymer shall form the matrix and the acidic-pH sensitive polymer shall be the component embedded in the hydrophobic matrix.

Typically the ratio between the hydrophobic polymer and the “pH sensitive” polymer (such as Eudragit E) is from about 5:1 to about 1.5:1, yet further preferably from about 3:1 to about 2:1.

It is important to note that the term weight ratio between the hydrophobic polymer and the pH sensitive polymer is the same in the liquid precursor composition, and in the dry film.

The term “therapeutic agent suitable for the treatment or prevention of a disorder or pathological condition” excludes agents which are intended to prevent, treat, ameliorate, or diminish altogether, oral disorders.

The term “oral disorders” includes any oral-related conditions and disorders including conditions that are directly related and associated with oral biofilms, dental and periodontal diseases (such as plaque, dental caries, gingivitis, periodontal diseases, root canal infections, tooth extractions, tooth hypersensitivity, viral infections, xerostomia, burning mouth, ulcers, candidiasis, tumours, aphthous, ulceration, abscesses, stomatitis, halitosis, dry mouth, salivary gland disfunction and including dental esthetics (tooth whitening).

In particular the therapeutic agent is selected from an antibiotic agent, an antibacterial agent, an antiseptic agent, an antifungal agent, an anti-viral agent, a bone and/or tissue growth factor agent, an anti-tumor agent, an anti-inflammatory agent, anti biofilm agent, an anti protozoa agent, hormones, enzymes, genes, anti irritation, anti pain, local analgestic, anti histamines, anti cancer, anastetic, peptides or plasmids agents.

Examples of antibiotic agents include, but are not limited to tetracycline derivatives, penicillin derivatives, macrolides derivatives, cephalosporin derivatives, lincosamides derivatives and glycopeptides derivatives, aminoglycyclitols derivatives, quinolones derivatives, sulfonamides derivatives, beta lactamase, chloraphenicol, macrolise, bacitracin, clindamycin, lincomycin, polymyxin, vancomycin, gentamycin

The term “antibacterial agent” includes any agent capable of killing bacteria.

Examples of an antiseptic agents, include, but are not limited to bacteriocidal quaternary ammonium salt such as cetylpyridinium chloride or benzalkonium chloride or chlorhexidine, or triclosan, or phenols derivatives, or polyphenols, or amino fluoride, inorganic salts of fluoride, or silver salts, or oxidative agents, or antiseptic volatile oils, herbal antiseptics or other bactericidal agent such as camphorated p-Chlorophenol (CPK) or iodine derivatives

Examples of antifungal agents include, but are not limited to polyenes, Nystatin, amphotericin, imidazoles, tinactin, clotrimazole, miconazole, ketonazole, triazoles, fluconazole and itraconazole, griseosulvine.

Examples of anti-viral agents include, but are not limited to acyclovir, amamatadine, diolamosine, famciclovir, foscaruet, ganciclovir, ribavirin, rimantadine, stavudine, zalcitabine, and zioloudine.

Examples of bone and/or tissue growth factor agents include, but are not limited to Bone Morphogenetic Proteins (BMPs), cytokines, simvastatine, IGF and FGF.

Examples of anti-inflammatory agents include, but are not limited to Steroidal and non-steroidal anti-inflammatory agents, include but not limited to: diclofonate, emoprofen, celecoxid, etodolat, inomethacine, laproxene, ketoprophen, rofecoxib, dexamethazone, prenisolone, betamethazone, mometatazone, hydrocortizole, triamcinolone acetonide, flumtazone, methyl prednizolone, pheylbutazone,

Examples of anti biofilm treatment agents include, but are not limited to active agents below the minimal effective concentration; enzymes degrading the matrix of the biofilm as proteolytic enzymes, carbohydrate degradation enzymes, surfactants and hydrophilic/hydrophobic agents, herbal extracts and anti quorum sensing agents acting as anti biofilm agents.

Interfering in cell cell/bacteria bacteria communications include, but are not limited to, active agents below the minimal effective concentration or specific agents.

Anti quorum sensing treatment agents include, but are not limited to herbal extracts e.g. garlic, furanones, homo serine lacton analogues, AI-2 analogues, competence stimulating peptides (CSP) analogues.

According to specific preferred embodiments, as can be seen in the examples below, the therapeutic agent is an antibacterial agent and/or an antifungal and/or steroid anti inflammatory agent or any of the listed agents above separately or together

The composition of the invention may contain any number of the agents. These may include, but are not limited to any combinations of the above, between the agents in the different groups and with in one group. Please write it as you feel is the best that we can use combinations

More specifically, the therapeutic agent is selected from triclosane, chlorhexidine-diacetate (CHX), clotrimazole and cetylpyridium-chloride (CPC).

The composition of the invention may additionally contain any number of biocompatible additives. These may include, but are not limited to, a plasticizer (such as polyethylene glycol, dibutyl phthalate glycerol or Triacetine), and thickeners such as hydroxyl propyl cellulose, hydroxy propyl methyl cellulose.

The liquid precursor composition described herein is capable of forming upon solidification thereof a matrix made of at least one hydrophobic polymer, having embedded within the at least one acidic-pH sensitive polymer and the at least one therapeutic agent.

The solidification of the liquid precursor of the invention into a solid matrix film can take place naturally by allowing the solvent to evaporate or can be facilitated by applying gentle heated air flow to the mouth.

The obtained matrix, formed by the solidification of the liquid precursor composition, forms a sustained release formulation suitable for the treatment and/or prevention of a variety of disorders.

Thus, according to another aspect of the invention, there is provided a medical device, that is to be inserted in or placed on a body of a subject, this device being coated by a sustained release formulation comprising a matrix made of at least one hydrophobic polymer, having embedded within at least one acidic-pH sensitive polymer and at least one therapeutic agent suitable for the treatment and/or prevention of a disorder or pathological condition, excluding oral disorders, such that the weight ratio between the at least one hydrophobic polymer and the at least one acidic-pH sensitive polymer is larger than 1.

Typically the diseases or disorders that are to be prevented or treated in accordance with the present invention are associated with a reduced pH—especially diseases and disorders caused by infectious microorganisms.

As detailed hereinabove, preferably this ratio ranges from about 5:1 to about 1.5:1, yet further preferably from about 3:1 to about 2:1.

As further detailed hereinabove, examples of such medical devices are: catheters (including urinary catheters), stents (including urinary stents), defibrillators, pacemakers, pumps, electrodes, artificial joints, air-tubes, CVS, implants, heart valves, intrauterine devices, artificial joints, implants, feeding tubes, ventilation tubes, IV's and discharge tubes.

As further noted hereinabove, the acidic-pH sensitive polymer forms between 10% by weight to 40% by weight of the total weight of the matrix.

The formulation of the invention can take a number of forms, such as a film, a gel, a foam, a varnish, a dosage meter spray.

After being applied on the desirable surface, it forms a very thin coating on the surface onto which it has solidified, this layer ranging from a few microns to a few hundred microns.

Preferably, the coating thickness should range from about 30 microns to about 150 microns.

The term “sustained release formulation”—refers to a formulation (in the case in a solid form) that allows an active agent contained therein to transfer to the physiological surrounding over a prolonged period of time, typically of at least one day.

The sustained release properties of the formulations of the invention are maintained even at these thin coatings, ranging from 3 to 240 hours. As the release rate varies with the thickness of the SRD coating it can range from hours to days pending the thickness and the environments as noted hereinabove. The liquid precursor compositions of the present invention are composed of enough hydrophobic polymer, compared to the acidic-pH sensitive polymer (namely that the weight ratio between them is larger than 1), to enable the formation of a hydrophobic matrix in which the pH-sensitive polymer and the therapeutic agent, are embedded.

This matrix is then capable of keeping its sustained release properties on the hard surface in the oral cavity, for hours and days, even at relatively thin coatings pending on the above ratio and the environment and location.

Typically, for coatings ranging from 30 microns to 150 microns, the rate of release would range from 3 to 12 hours respectfully.

However, pending on the surface on which the composition is applied, the thickness and the location, the release rated can be tailored to be at least 3 days.

When the pH is neutral, the formulation of the invention maintains a graduate slow release rate of the therapeutic agent. As explained hereinabove, when pathologies develop (for example when bacterial infection effects the region), a pH decrease to about or below pH 6.0 occurs. In the acidic pH environment formed in this region, the acidic pH sensitive polymer (for example Eudragit E) is degraded, thereby increasing the release rate of the therapeutic agent from the matrix in which it is also embedded. It should be noted that even at extremely acidic pH not all of the therapeutic agent will be released at once (at a “burst”) due to the constant degradation rate of the hydrophobic polymer. The faster release rate of the therapeutic agent will continue until the pH increases again due to the cease of the pathological condition (for examples due to cease of the bacterial infection). This “sensor” effect is far better than a classic sustained release delivery system, in which the release is by a constant profile, regardless of the environmental feedback.

Typically, the formulation in the solid form is resistant to some degree to erosion. It should be emphasized that according to the invention the release rate is not constant but changes in response to the changes in the environment, in particular due to pH changes. The lower the pH (indicative of the presence/deterioration of a disease or a disorder), the faster is the release rate and vise versa—making the release dependent on the severity/existence of the condition or the disorder.

Given these advantages, namely the ability to “sense” disorders associated with low pH, the sustained and prolonged release of the therapeutic agent, and the sensitivity of the system to the success of the treatment (and rising of the pH), the formulations described herein are especially suitable for the treatment of infectious disorders and conditions.

Thus, according to yet another aspect of the invention, there is provided a method for treating, preventing, ameliorating or eliminating altogether at least one disorder or one pathological condition, this method comprising applying the liquid precursor compositions of the invention on a surface to be treated, and allowing the composition to solidify on this surface, thereby forming a film; wherein the surface is a surface on the body and/or area and/or organ of said subject to be treated and/or is a surface of a medical device to be placed in or on a body of a subject to be treated.

The term “film” includes both a coating (or coat) and a varnish.

Thus, according to another aspect of the invention, there is provided a method for applying the above liquid precursor compositions of the invention, on a device to be placed in or on the body of a subject, and allowing the liquid composition to solidify, thereby forming a film for sustained release of the therapeutic agent onto the device.

The therapeutic agent used in this method is as described above, and is preferably an anti infective agent, such as bacterial agent, antiviral agent, anti protozoa agent, anti fungal agent and anti inflammatory agent.

The application may be by applying the composition to the device (by brushing, spraying etc) or by immersing the device in the liquid precursor composition of the invention.

The liquid precursor composition and the method of the invention are applicable for human or veterinary use.

### DETAILED DESCRIPTION OF THE INVENTION

### Experimental

### Materials and Methods

Active Agents
Chlorhexidine-Diacetate (CHX), Cetylpyridinium-Chloride (CPC), Clotrimazole and Triclosane were all obtained from Sigma-Aldrich, St. Louis, USA.

Excipients
Eudragit E PO (Rohm Gmbh, Germany)
Sodium Layryl Sulfate (SLS) (Riedel de Haen, Sigma-Aldrich Gmbh, Germany)
Ethylcellulose—(EC) (Ethocel Premium N 100, Dow Chemical Company Russelville, USA)
Ethanol (J.T. Baker Deventer Holland)
Polyethylenglycol 400(PEG 400) (Schuchardt Hohenbrunn Germany)
Sodium acetate 3 hydrate
1-Heptanesulfonic acid sodium salt (J.T. Baker NJ USA)

Additional Ingredients
Trizma Base (2-amino-2-(hydroxymethyl)-1,3-propanediol)—(Sigma-Aldrich, St. Louis, USA)
Phosphate buffer USP pH=6.8
Phosphate buffer USP pH=5.0

### Example 1

### Preparation of pH Sensitive Liquid Precursor Compositions Containing Clotrimazole, and Applications Thereof

I. Preparation of Liquid Precursor Composition:

PEG400 was weighted into the ethanol. Then, the dry powders of the hydrophobic polymer (Ethyl Cellulose) and the pH-sensitive polymer (Eudragit-E) were slowly added as dry powders to ethanol, and vigorously stirred for about 30 minutes until complete dissolution. Then, the clotrimazole (active agent) was added while continuously stirring.

II. Preparation of Film from the Liquid Precursor Composition:

The liquid precursor composition obtained in part I was poured (15 ml) on Teflon dishes (10.5 cm diameter) in a drying room and dried for about 4 hours. The obtained film was 0.230 mm thick.

Table 1 below shows the clotrimazole sample prepared, showing its composition both in the dry film and in the liquid precursor composition.

TABLE 1

| | | | % weight in |
|---|---|---|---|
| | | % weight in | liquid precursor |
| Formulation | Ingredient | dry film | composition |
| Clotrimazole-1 | Clotrimazole | 52.18 | 5.303 |
| | Ethyl Cellulose (EC) | 39.13 | 3.98 |
| | PEG 400 | 4.98 | 0.508 |
| | Eudragit E | 17.99 | 1.834 |
| | Ethanol | | 88.38 |

Release Rate Experiment:

Determining the clotrimazole release rate from the films was conducted by first placing the films in 350 ml glass vessels, containing Trizma base buffer (50 mM) with 0.2% SLS (sodium lauryl sulphate) at pH=5.0 and 6.8 and 50 cpm, at 37° C.

Then, 1 cc samples were taken from the glass vessels at pre-determined intervals (each hour from 1 to 8 hrs). The released clotrimazole concentration was measured spectrophotometrically at 206 nm (Uvikon 933: Kontron Instruments). The concentration of the CHX was calculated according to a reference curve.

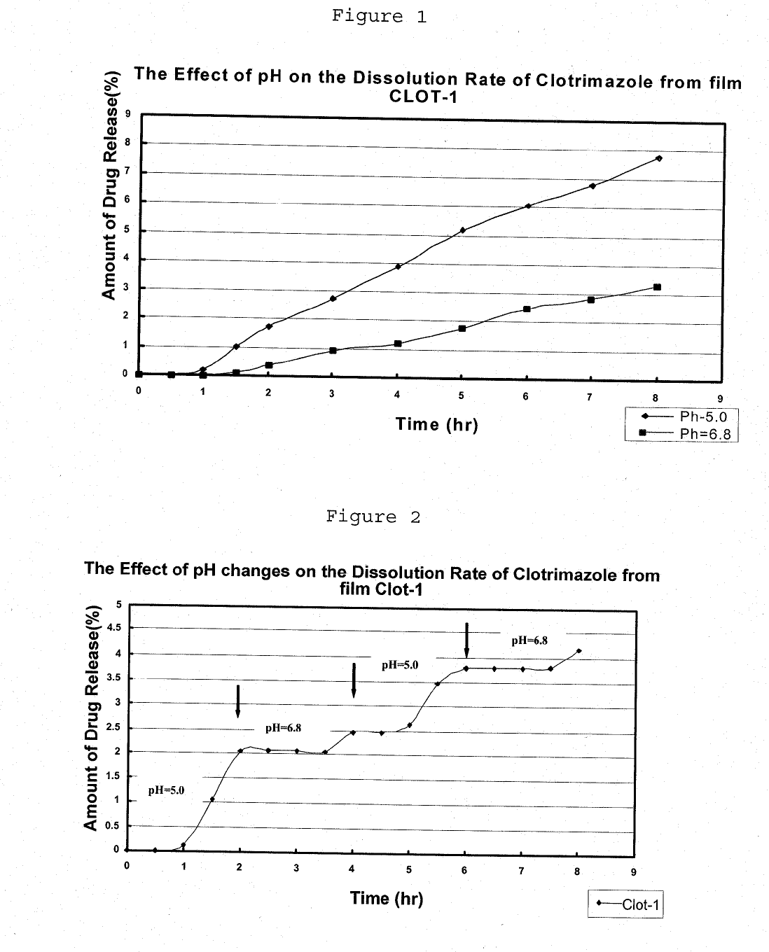

FIG. 1 shows the clotrimazole release rate (as % of the initial amount in film) with time (1-8 hours), for two different pHs: 5.0 and 6.8. As is clear from the figure, the release rate at pH 5.0 was much faster than at pH 6.8

In a different experiment, the films were kept at a pH 5.0 buffer for 2 hours, then kept at a pH 6.8 buffer for 2 hours, then at a pH 5.0 buffer for 2 hours and again at a pH 6.8 buffer for 2 hours. Samples were taken at similar intervals. The results are shown in FIG. 2, which shows the clotrimazole release rate (as % of the initial amount in film) with time (1-8 hours), for this pH change profile. It again demonstrates that pH of 6.8 retards the rate of release from the pH sensitive SRD.

### Example 2

### Preparation of pH Sensitive Liquid Precursor Compositions Containing Chlorhexidine-Diacetate (CHX), and Applications Thereof

I. Preparation of Liquid Precursor Composition:

The liquid precursor composition was prepared as described in Example 1 (part I), replacing the clotrimazole by chlorhexidine-diacetate (CHX).

II. Preparation of Film from the Liquid Precursor Composition:

The liquid precursor composition obtained in part I was poured (21 ml) on Teflon dishes (10.5 cm diameter) in a drying room (37° C.) and dried for about 4 hours. The obtained film was 0.120 mm thick.

Table 2 below shows the CHX sample prepared, showing its composition both in the dry film and in the liquid precursor composition.

TABLE 2

| | | | % weight in |
|---|---|---|---|
| | | % weight in | liquid precursor |
| Formulation | Ingredient | dry film | composition |
| CHX-1 | CHX | 47.4 | 4.5 |
| | Ethyl Cellulose (EC) | 32.6 | 3.1 |
| | PEG 400 | 5.3 | 0.5 |
| | Eudragit E PO | 14.7 | 1.4 |
| | Ethanol | | 90.5 |

Release Rate Experiment:

Determining the CHX release rate from the films was conducted by first placing the films in 100 ml glass vessels, containing phosphate buffer at pH=5.0 and 6.8 and 50 cpm, at 37° C.

Then, 1 cc samples were taken from the glass vessels at pre-determined intervals (each hour from 1 to 8 hrs).

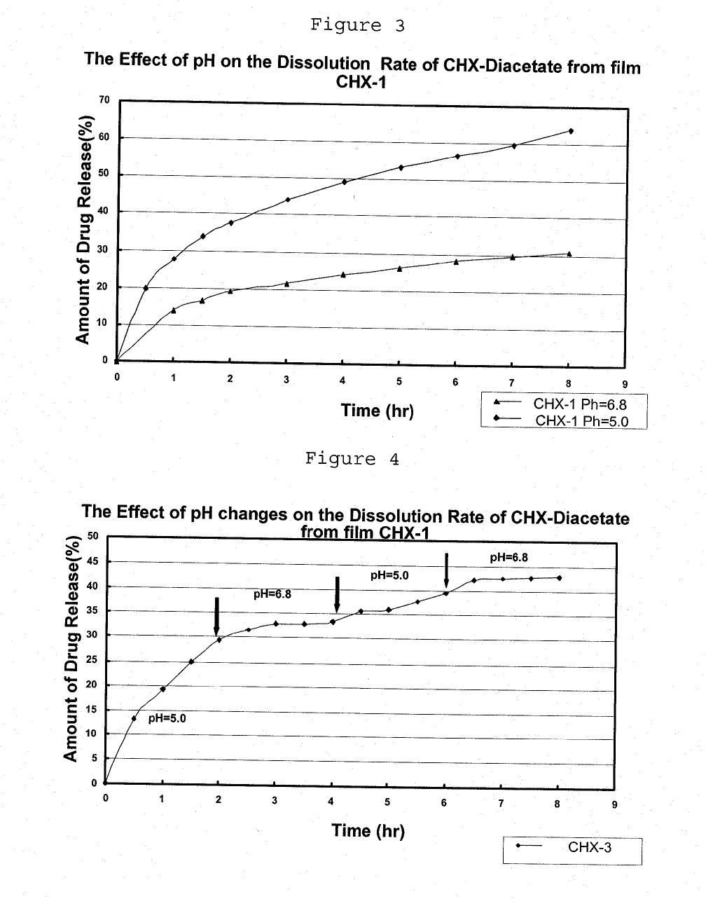

The released CHX concentration was measured spectrophotometrically at 260 nm (Uvikon 933: Kontron Instruments). The concentration of the CHX was calculated according to a reference curve. FIG. 3 shows the CHX release rate (as % of the initial amount in film) with time (from 1-8 hours) at two different pHs: 5.0 and 6.8, again showing a higher release rate at pH 5.0.

In a different experiment, the films were kept at a pH 5.0 buffer for 2 hours, then at a pH 6.8 buffer for 2 hours, then at a pH 5.0 buffer for 2 hours and again at a pH 6.8 buffer for 2 hours. Samples were taken at similar intervals. The results are shown in 4, which shows the CHX-1 release rate (as % of the initial amount in film) with time (1-8 hours), for this pH change profile. As shown at each sampling interval in FIG. 4, the release rate of CHX at pH 5.0 was faster than at pH 6.8, also demonstrating that pH of 6.8 retards the rate of release from the pH sensitive SRD.

### Example 3

### Preparation of pH Sensitive Liquid Precursor Compositions Containing Triclosane, and Applications Thereof

I. Preparation of Liquid Precursor Composition:

The liquid precursor composition was prepared as described in Example 1 (part I), replacing the clotrimazole by triclosane.

II. Preparation of Film from the Liquid Precursor Composition:

The liquid precursor composition obtained in part I was poured (15 ml) on Teflon dishes (10.5 cm diameter) in a drying room and dried for about 4 hours. The obtained film was 0.177 mm thick.

Table 3 below shows the triclosane sample prepared, showing its composition both in the dry film and in the liquid precursor composition.

TABLE 3

| | | | % weight in |
|---|---|---|---|
| | | % weight in | liquid precursor |
| Formulation | Ingredient | dry film | composition |
| Triclosane-1 | Triclosane | 34.0 | 3.3 |
| | Ethyl Cellulose (EC) | 40.2 | 3.9 |
| | PEG 400 | 12.4 | 1.2 |
| | Eudragit E | 13.4 | 1.3 |
| | Ethanol | | 90.3 |

III. Determining the Release Rate of Triclosane from the Film of Part II:

Determining the triclosane release rate from the films was conducted by first placing the films in 100 ml glass vessels, containing Trizma base buffer (50 mM, +10% SLS (sodium lauryl sulphate) at pH-5.0 and 6.8 at 50 rpm, at 37° C.

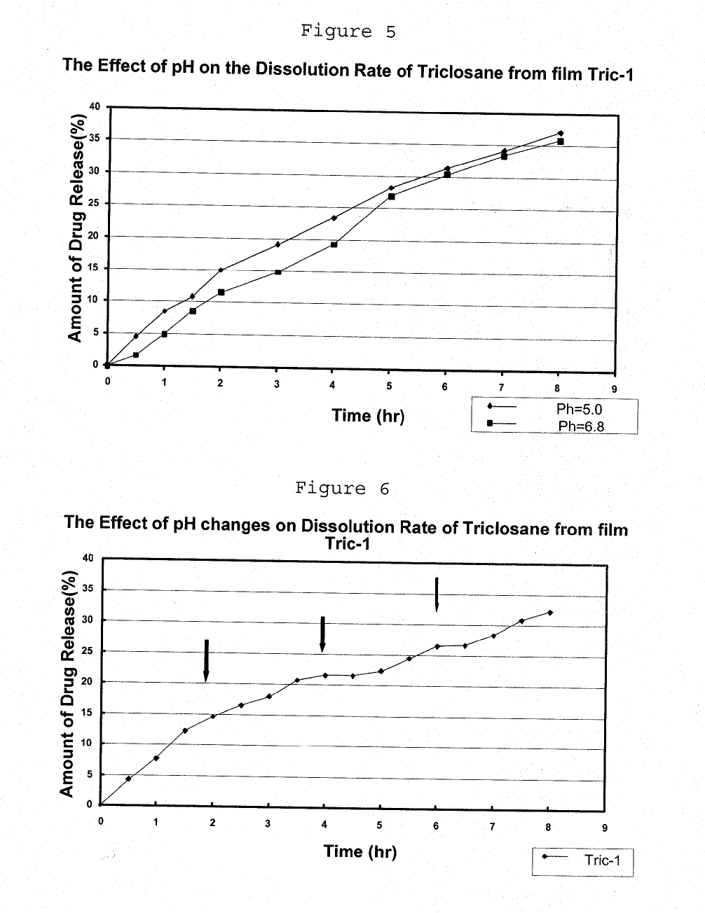

Then, 2 cc samples were taken from the glass vessels at pre-determined intervals (each hour from 1 to 8 hrs). The released triclosane concentration was measured spectrophotometrically at 280 nm (Uvikon 933: Kontron Instruments). The concentration of the triclosane was calculated according to a reference curve. FIG. 5 shows the triclosane (Tric-1) release rate as % of the initial amount in the film, with time (from 1-8 hours) at two different pHs: 5.0 and 6.8.

In a different experiment, the films were kept at a pH 5.0 buffer for 2 hours, then at a pH 6.8 buffer for 2 hours, then at a pH 5.0 buffer for 2 hours and again at a pH 6.8 buffer for 2 hours. Samples were taken at similar intervals. The results are shown in FIG. 6, which shows the Tric-1 release rate (as % of the initial amount in film) with time (1-8 hours), for this pH change profile.

As shown at each sampling interval of FIG. 6, the release rate of triclosane at pH 5.0 was significantly faster than at pH 6.8.

### Example 4

### Preparation of pH Sensitive Liquid Precursor Compositions Containing Cetylpyridinium-Chloride (CPC), and Applications Thereof

I. Preparation of Liquid Precursor Composition:

The liquid precursor composition was prepared as described in Example 1 (part I), replacing the clotrimazole by Cetylpyridinium-Chloride (CPC).

II. Preparation of Film from the Liquid Precursor Composition:

The liquid precursor composition obtained in part I was poured (15 ml) on Teflon dishes (10.5 cm diameter) in a drying room and dried for about 4 hours. The obtained film was 100-150 micron thick.

Table 4 below shows the CPC sample prepared, showing its composition in the liquid precursor composition.

TABLE 4

| | Sample CPC-1 | Sample CPC-2 | Sample CPC-3 |
|---|---|---|---|
| Ingredient | % in liquid precursor formulation | | |
| Cetylpyridinum | 10% | 15% | 20% |
| Chloride (CPC) | | | |
| Ethyl Cellulose | 5% | 5% | 5% |
| Eudragit E | 1% | 2% | 3% |
| Triacetine | 1% | 1% | 1% |
| (plasticizer) | | | |
| Ethanol | 83% | 77% | 71% |

### Example 5

### Comparing the Antibacterial Activity of Samples with and without pH-Sensitive Polymers

Ethyl-cellulose-based formulations with antimicrobial agents—Chlorhexidine (CHX) and Cetylpyridinium-Chloride (CPC) were prepared as detailed above in Examples 2 and 4, respectively either with the acidic pH sensitive polymer (Eudragit E) or without it. The duration of antibacterial bio-assay activity on S. mutans ATCC 27351 bacteria was tested by daily growth inhibition zone measurements around the formulations followed by transfer of the formulations to a newly plated agar media, until no inhibition was observed.

The compositions of the different tested liquid precursor compositions are given in Table 5 below.

TABLE 5

| | | | | | formula 3: | |
|---|---|---|---|---|---|---|
| | formula 1: | | | | direct | |
| | Controlled | | formula 2: | | dripping on a | |
| Ingredient | Release | | Placebo | | Wattman paper | |
| CHX | 2.5 | grams | None | | 0.07 | gr |
| (Active agent) | (47.2% in dry film) | | | | | |
| Ethyl | 1.74 | gr | 1.74 | gr | None | |
| cellulose | (32.1% in dry film) | | | | | |
| Eudragit E | 0.5 | gr | 0.5 | gr | None | |
| | (9.4% in dry film) | | | | | |
| PEG400 | 0.6 | gr | 0.6 | gr | None | |
| | (11.3% in dry film) | | | | | |
| Ethanol | 50 | ml | 50 | ml | 1.4 | ml |

### Results

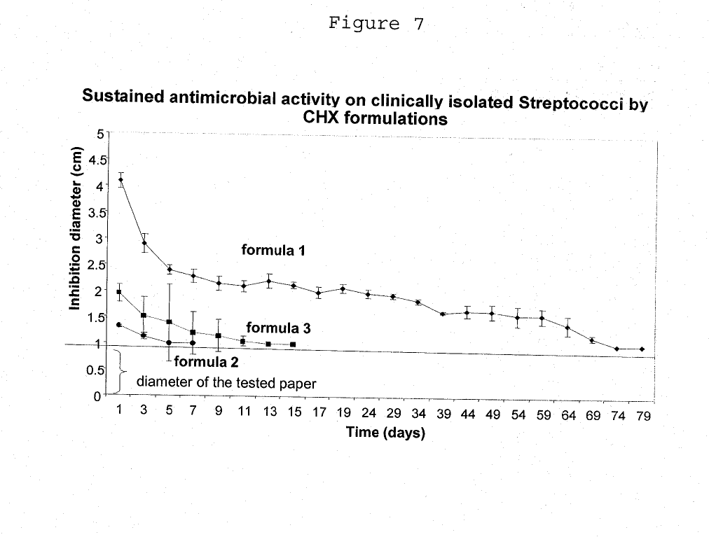

FIG. 7 shows the sustained antimicrobial activity on clinically isolated Streptococci by CHX formulations for formulas 1-3 detailed above, as a function of time (in days). As can be seen in FIG. 7, SRD containing CHX as an antimicrobial agent and a pH sensitive polymer Eudragit E exhibited the best prolonged antibacterial activity in-vitro, (for over 79 days) on S. mutans.

### Example 6

### Mastitis, Disease of the Udder's Cow

The SRV was applied on udder's cow, dried and the tissue was placed on agar for bioassay using three different bacteria as described below.

The SRV is composed of Ethyl cellulose (5 gr), Klucel EF (3.5 gr), PEG 400 0.2 g, Eudragit E (0.5 g) and CHX diacetate 0.1% dissolved in 100 cc of ethanol

Bioassays:

The release rate of the coated catheters/cow's udder was examined by Bioassay as follows. The examined pieces were coated with the varnish and dried at room temperature. The coated pieces were placed on agar plate pre seeded with various bacteria. After incubation at 37 C the zone of inhibition was measured around the coated object and it was teased to a new pre seeded agar plate:

Inhibition zones around SRV coated cow's adder (Bioassay)

### Example 7

### Catheters Coated with pH Sensitive SRV

Catheters were coated with SRV containing CHX in a similar formulation as in Example 1.

Bioassays were conducted as described above.

Area of Inhibition (mm²)

TABLE 6

| | Day | | | | | |
|---|---|---|---|---|---|---|
| | 1 | Day 2 | Day 3 | Day 4 | Day 5 | Day 6 |
| Proteus 4434 | 8.8 | 3.6 | 4.4 | 6.9 | 8.7 | 2.7 |
| E. Coli 5094 | 7.1 | 7.5 | 8.1 | 11.5 | 3.0 | 2.6 |
| E. Coli 5038 | 8.7 | 9.1 | 6.9 | 6.5 | 5.7 | 5.0 |
| Staphylococcus | 12.2 | 7.2 | 10.8 | 4.3 | 6.2 | 3.5 |
| intermedius 1433 | | | | | | |
| Staphylococcus | 10.7 | 8.2 | 6.6 | 8.3 | 7.6 | 2.3 |
| intermedius 2091 | | | | | | |
| Staphylococcus | 11.3 | 11.6 | 8.5 | 10.5 | 5.8 | |
| intermedius 1219 | | | | | | |

### Example 8

### Treated Urinary Catheters in Dogs

Thirteen dogs had a coated urinary catheter placed (study group) and 13 dogs had an untreated urinary catheter (control group). Presence and intensity of biofilm formation on the urinary catheter was assessed and compared between the groups by evaluating colony forming unit (CFU) count of biofilm bacteria, and semi-quantitatively using confocal and electron microscopy.

All dogs were treated with antibiotics during their hospitalization due to reasons unrelated to the urinary condition.

Urinary catheter was left in place in the study group for a median of 72 hours (range 24-168 hours) and in the control dogs for 36 hours (24-144 hours) with no statistically significance difference between the groups (P=0.19). None of the dogs presented any side effect that could have been attributed to the presence of the urinary catheter.

Urine cultures at the time of urinary catheter placement were all negative. Urine cultures just prior to catheter removal were available in 12 and 9 dogs of the control and the study group, respectively. The proportion of dogs with positive urine culture tended to be lower in the study compared to the control group (1/8, 11% vs. 6/12, 50%, P=0.06).

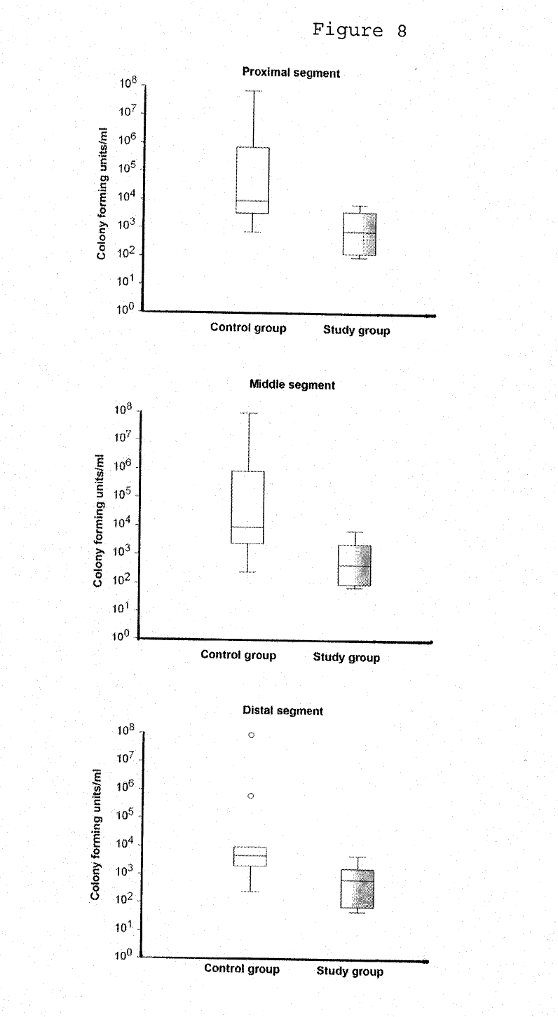

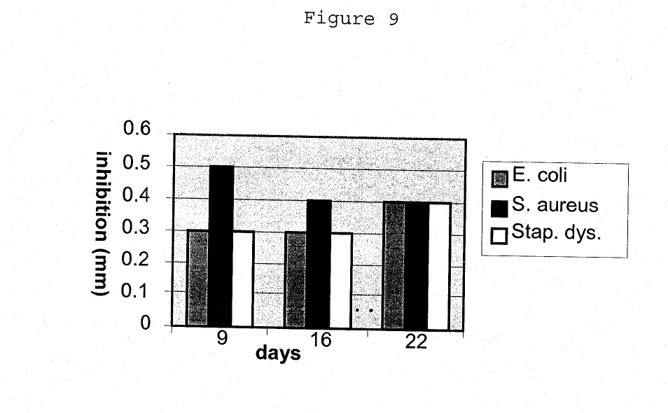

Median CFU count of biofilm bacteria at the proximal portion of the urinary catheters was significantly lower in the study compared to the control group [median 125 CFU/ml (range, 0-7.5×10³ CFU/ml vs. median, 10×10⁵CFU/ml (range, 0.75-7.5×10⁷ CFU/ml), P<0.001] (FIG. 8). Median CFU of biofilm bacteria at the middle portion of the urinary catheters was significantly lower in the study compared to the control group [median 75 CFU/ml (range, 0-7.5×10³ CFU/ml) vs. median, 10×10⁵ CFU/ml (range, 0.25-1×10⁸ CFU/ml), P<0.001]. Median CFU of biofilm bacteria at the distal portion of the urinary catheters was also significantly lower in the study compared to the control group [median 50 CFU/ml (range, 0-5×10³ CFU/ml) vs. median, 5×10³ CFU/ml (range, 0-8.7×10⁷ CFU/ml), P<0.001] (FIG. 8).

The CFU count was higher in bacteria immobilized on the proximal part compared to the middle and distal part in 35% and 54%, respectively; they were equal in 54% and 35%, respectively while in 11% the CFU counts were higher in the middle and distal part compared to the proximal part.

The proportion of dogs that were classified as none/mild, based on the results of the CLSM, was significantly higher in the study compared to the control group in all part of the urinary catheter (Table 7). The proportion of dogs that were classified as none/mild based on the results of the scanning electron microscopy was also significantly higher in the study compared to the control groups in all part of the urinary catheter (Table 8).

The electron microscopic examination revealed presence of crystals on some of the urinary catheters. In the proximal part of the catheter, the proportion of crystals tended to be lower in the study compared to the control group (7.7% vs. 46.2%, respectively, P=0.07). In the middle part of the catheter proportion of crystals was not statistically different between the study and the control group (7.7% vs. 15.4%, respectively, P=0.13). In the distal part of the catheter proportion of crystals was lower in the study compared to the control group (16.7% vs. 66.7%, respectively, P=0.04).

TABLE 7
Degree of bacteria present on the different part of the
urinary catheter as evaluated by confocal microscopy

| | | Study | Control | |
|---|---|---|---|---|
| | | group | group | |
| | Florescence | (n, %) | (n, %) | P value |
| Proximal | None/Mild | 10 (76.9) | 2 (15.4%) | 0.0016 |
| part | Moderate/Severe | 3 (23.1%) | 11 (84.6%) | |
| Middle part | None/Mild | 11 (84.6%) | 5 (38.5%) | 0.015 |
| | Moderate/Severe | 2 (15.4%) | 8 (61.5%) | |
| Distal Part | None/Mild | 11 (84.6%) | 6 (46.2%) | 0.039 |
| | Moderate/Severe | 2 (15.4%) | 7 (53.8) | |

TABLE 8
Degree of bacteria present on the different part of the
urinary catheter as evaluated by electron microscopy

| | | Study | Control | |
|---|---|---|---|---|
| | | group | group | |
| | Florescence | (n, %) | (n, %) | P value |
| Proximal | None/Mild | 13 (100%) | 9 (69.2%) | 0.030 |
| part | Moderate/Severe | 0 (0.0) | 4 (30.8%) | |
| Middle part | None/Mild | 13 (100%) | 8 (61.5%) | 0.013 |
| | Moderate/Severe | 0 (0.0) | 5 (38.5%) | |
| Distal Part | None/Mild | 13 (100%) | 9 (69.2%) | 0.030 |
| | Moderate/Severe | 0 (0.0) | 4 (30.8%) | |

1. A liquid precursor composition adapted for application on a on a desired surface, said composition comprising:
a. at least one therapeutic agent suitable for the treatment or prevention of a disorder or pathological condition, wherein said disorder or pathological condition excludes oral disorders,
b. at least one acidic-pH sensitive polymer,
c. at least one hydrophobic polymer, and
d. a pharmaceutically acceptable volatile solvent, wherein a weight ratio between said at least one hydrophobic polymer and said at least one acidic-pH sensitive polymer is larger than 1.

2. The composition of claim 1, wherein said weight ratio between said at least one hydrophobic polymer and said at least one acidic-pH sensitive polymer ranges from about 5:1 to about 1.5:1.

3. The composition of claim 1, being capable of forming upon solidification thereof a matrix made of said at least one hydrophobic polymer, having embedded within said at least one acidic-pH sensitive polymer and said at least one therapeutic agent.

4. The composition of claim 1, wherein said surface is a surface of a medical device that is to be inserted in or placed on a body of a subject.

5. The composition of claim 4, wherein said medical devices is selected from catheters, stents, defibrillators, pacemakers, pumps, electrodes, artificial joints, air-tubes, CVS, implants, heart valves, intrauterine devices, artificial joints, implants, feeding tubes, ventilation tubes, IV's and discharge tubes.

6. The composition of claim 1, wherein said surface is an external surface of body tissue.

7. The composition of claim 6, wherein said body tissue is selected from: soft tissues, skin, mucosa, nails, hoofs and udder.

8. The composition of claim 1, wherein said hydrophobic polymer is selected from: copolymer hydrogels of hydroxymethyl methacrylate (HEMA) and methylmethacrylate (MMA), Ethyl cellulose (EC), poly(acrylic acid), poly(methyl vinyl ether co-maleic anhydride), pol(ethylene oxide), karya gum, poly(hydroxyethylmethacrylate), Silicone rubber, polyethylene, polylactic acid, polymethylmethacrylate, polyvinyl chloride, polyvinyl acetate, and polyurethane.

9. The composition of claim 1, wherein said therapeutic agent is selected from an antibiotic agent, an antibacterial agent, an antiseptic agent, an antifungal agent, an anti-viral agent, a bone and/or tissue growth factor agent, an anti-tumor agent, an anti-inflammatory agent, anti biofilm agent, an anti protozoa agent, hormones, enzymes, genes, anti irritation, anti pain, local analgestic, anti histamines, anti cancer, anastetic, peptides or plasmids agents

10. The composition of claim 9, wherein said therapeutic agent is selected from triclosane, chlorhexidine-diacetate (CHX), clotrimazole and cetylpyridium-chloride (CPC).

11. The composition of claim 1, wherein said acidic-pH sensitive polymer is selected from Eudragit E, acrylic compounds or any compounds containing primary, secondary or tertiary amine groups.

12. A medical device that is to be inserted in or placed on a body of a subject, said device being coated by a sustained release formulation, comprising a matrix made of at least one hydrophobic polymer, having embedded within at least one acidic-pH sensitive polymer and at least one therapeutic agent for the treatment or prevention of a disorder or pathological condition, wherein said disorder or pathological condition excludes oral disorders, such that the weight ratio between said at least one hydrophobic polymer and said at least one acidic-pH sensitive polymer is larger than 1.

13. The device of claim 12, being selected from catheters, stents, defibrillators, pacemakers, pumps, electrodes, artificial joints, air-tubes, CVS, implants, heart valves, intrauterine devices, artificial joints, implants, feeding tubes, ventilation tubes, IV s and discharge tubes.

14. The device of claim 12, wherein said acidic-pH sensitive polymer forms between 10% by weight to 40% by weight of the total weight of said matrix.

15. The device of claim 12, being in a form of a film having a thickness range from about 30 microns to about 150 microns.

16. The device of claim 12, having a release rate of said therapeutic agent, ranging from 3 to 240 hours.

17. A method for treating, preventing, ameliorating or eliminating altogether at least one disorder or one pathological condition, said method comprising applying the liquid precursor compositions of claim 1 on a surface to be treated, and allowing said composition to solidify on said surface, thereby forming a film; wherein said surface is a surface on the body and/or area and/or organ of said subject to be treated and/or is a surface of a medical device to be placed in or on a body of a subject to be treated.

18. A method for applying the liquid precursor composition of any claim 1 on a device to be placed in or on a body of a subject, and allowing said liquid composition to solidify, thereby forming a film for sustained release of a therapeutic agent onto said device.

19. The method of claim 18, wherein said composition is applied onto said device by brushing and/or spraying; or wherein said composition is applied by immersing said device in said liquid composition.
